(19) United States
(12) Patent Application Publication
(10) Pub. No.: US 2008/0148461 A1
(43) Pub. Date: Jun. 26, 2008

(54) Eye-covering assembly

(76) Inventors: Bahman Guyuron, Lyndhurst, OH (US); Jamie Horvath, Lakewood, OH (US)

Correspondence Address: Cynthia S. Murphy;Renner, Otto, Boisselle & Sklar, 1621 Euclid Ave. - 19th Floor, Cleveland, OH, 44115, US

(21) Appl. No.: 12/001,268

(22) Filed: Dec. 11, 2007

Related U.S. Application Data
(60) Provisional application No. 60/869,444, filed on Dec. 11, 2006.

(51) Int. Cl. A61F 9/00; A61F 9/02

(52) U.S. Cl. 2/15

(57) ABSTRACT

An eye-covering assembly (10) comprises a film layer (20) and a rim layer (30) having an inner surface (32) which adheres to the skin of the user's face. The inner and outer surfaces of the film/rim layers (20/30) are planar and parallel when the eye-covering assembly is in a pre-use relaxed state. The inner and outer surfaces of the film/rim layers (20/30) are curved and the rim layer (30) is compressed in certain regions, when the eye-covering assembly is in an in-use conforming state whereat it conforms to the contour of an eye-surrounding portion of a user's face.

### RELATED APPLICATION

This application claims priority under 35 U.S.C. §119(e) to U.S. Provisional Patent Application No. 60/869,444 filed on Dec. 11, 2006. The entire disclosure of this provisional application is hereby incorporated by reference.

### GENERAL FIELD

This disclosure relates generally to an eye-covering assembly which can be used to cover, shield, and/or protect a user's eye for desired period of time.

### BACKGROUND

An eye-covering assembly can be used to cover, shield, and/or protect, a user's eye for a desired period. For example, eye-covering assemblies can be worn to mitigate the discomforts experienced when adequate moisture cannot be retained in the membranes of the eyes. Such “dry-eye” discomforts can occur when a person does not completely close his/her eyes when sleeping, is continuously exposed to dry (or recirculated) air, is recovering from an eye surgery/injury, is caught in a coma, and/or is placed under anesthesia. An eye-covering assembly can create a moisture barrier around the person's eye to prevent the evaporation of membrane moisture. Other uses for eye-coverings include, for example, protecting eyes from light rays for sensitivity or suntanning reasons, shielding eyes from scratches, lasers, splashes impacts or other traumas, and/or sealing eyes from water when swimming or bathing. If both eyes are to be covered, a pair of eye-covering assemblies can be used.

### SUMMARY

An eye-covering assembly can be constructed for economic-production, easy-application, and/or compact compilation thereby providing numerous advantages. For example, it is not cost-prohibitive for the eye-covering to be a single-use item which is discarded after each use, thereby eliminating the need for cleaning and sterilization. Also, a dry-eye suffer could use a pair of the eye-coverings on a nightly basis without unreasonable expense. Because the eye-coverings can be conveniently stored and/or supplied, and do not require special skills to apply, this opens the opportunity of distributing and/or selling in some commercial settings, such as airports and/or hotels in dry climates. Facilities with tanning salons, sauna, pools, and/or other spa services could offer the eye-covering assemblies to patrons without the hassle (or liability) of post-use cleaning. In disaster, combat, or other hostile settings, such an eye-covering assembly can be godsend for the treatment of eye injuries. Sophisticated medical facilities would probably also welcome the economic, inventory, and application advantages of the eye-covering assembly, as well as the option of sending the patient home with such coverings after discharge. These and other features of the eye-covering assembly are fully described and particularly pointed out in the claims. The following description and annexed drawings set forth in detail a certain illustrative embodiment, this embodiment being indicative of but one of the various ways in which the principles may be employed.

### DRAWINGS

FIGS. 1A-1D are various plan views of the eye-covering assembly in a pre-use relaxed state.

FIG. 1E is a sectional view of the eye-covering assembly as seen along line 1E-1E in FIG. 1B.

FIGS. 1F and 1G are close-up views of circled portions of the eye-covering assembly in FIG. 1E.

FIG. 2A is a front view of the eye-covering assembly being worn by a user.

FIGS. 2B and 2C are sectional views of the eye-covering assembly as seen along lines 2B-2B and 2C-2C in FIG. 2A, respectively.

FIGS. 3A-3E are views of another eye-covering assembly.

### DETAILED DESCRIPTION

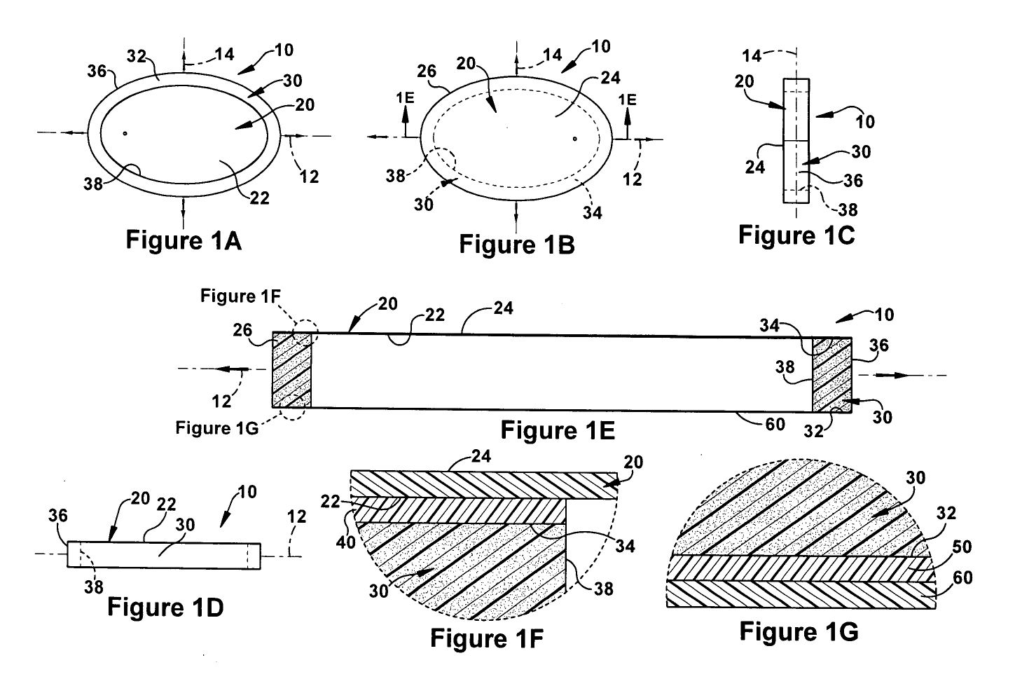

Referring now to the drawings, and initially to FIGS. 1A-1G, an eye-covering assembly 10 is shown in a pre-use relaxed state. The eye-covering assembly 10 can be worn to create a moisture barrier (e.g., to relieve dry-eye symptoms), to protect eyes from light rays for sensitivity or suntanning reasons, to shield eyes from scratches, lasers, splashes impacts or other traumas, and/or to seal eyes from water when swimming or bathing. Essentially, the assembly 10 may be used whenever a person's eye needs to be covered in a goggle-like manner.

The eye-covering assembly 10 can have a film/rim layered construction comprising a film layer 20 and a rim layer 30 which can each be one-piece layer (i.e., formed in one piece without seams or joints.) The film layer 20 has an inner surface 22, an outer surface 24, and an outside perimeter 26. The rim layer 30 has an inner surface 32, an outer surface 34, an outside perimeter 36, and an inside perimeter 38. The inner surface 22 of the film layer 20 is bonded to the outer surface 34 of the rim layer 30, and the outer surface 24 of the film layer 20 forms the outermost surface of the eye-covering assembly 10. The inner surface 32 of the rim layer 30 is adhered to the user's face during use of the assembly 10. The outside perimeters 26 and 36 define the overall shape of the eye-covering assembly 10 and the inside perimeter 38 of the rim layer 30 defines a window-like opening.

When the eye-covering assembly 10 is in the relaxed state shown in FIGS. 1A-1G, the inner/outer surfaces 22/24 of the film layer 20 and the inner/outer surfaces 32/34 of the rim layer 30 are planar and parallel. Also, the film layer 20 has a uniform thickness and the rim layer 30 has a non-compressed uniform thickness when the assembly 10 is in the relaxed state. This relaxed geometry can allow for a compact compilation of a plurality of the eye-covering assemblies 10 which can foster convenient stacking, storing, shipping, and/or supplying.

The outside perimeter 26 of the film layer 20 and the outside perimeter 36 of the rim layer 30 can have the same shape and/or can be the same size. In any event, the film/rim shape(s) can be viewed as having a nose-temple axis 12 and a brow-cheek axis 14 and, the dimension in the nose-temple direction can be greater than the dimension in the brow-cheek direction. For example, the film/rim layers 20/30 can have an elliptical shape. If the shape of the film/rim layers is symmetrical about the nose-temple axis 12, the orientation of the eye-covering assembly 10 will be invertible. If the shape of the film/rim layers 20/30 is symmetrical about the brow-cheek axis 14, the eye-covering assembly 10 can be used in either the left eye or the right eye.

The film/rim layers 20/30 can be sized and shaped for positioning within the user's eye socket (as opposed to around the entire orbit) as this shape/size may less be likely to dislodge in certain situations. The film/rim layers can have a dimension in the nose-temple direction of about 1¼ inches to about 2¾ inches, of about 1½ inches to about 2½ inches, and/or of about 1¾ inches to about 2¼ inches. The film/rim layers 20/30 can have a dimension in the brow-cheek direction of about ½ inch to about 2 inches, about ¾ inch to about 1¾ inches, and/or about 1 inch to about 1½ inches.

The film layer 20 can be a flexible polymeric film such as a polyolefin, polyvinyl, polyethylene, polycarbonate, polypropylene, polystyrene, polyurethane, and/or acrylic film. The uniform thickness of the film layer 20 can be between about 0.0001 inch and 0.010 inch, between about 0.0010 inch and 0.007 inch, and/or between about 0.003 inch and about 0.005 inch. The film layer 20 can include one or more holes to prevent over-accumulation of moisture and/or to more comfortably release suction when un-adhering the covering 10 from the wearer's skin.

The film layer 20 can be a transparent, translucent, colored, light-filtering, and/or completely opaque layer. With non-opaque layers, light can pass through the window-like area of the film layer 20 defined by the inside perimeter 38 of the rim layer 30. Likewise, the person's eye can be visible through the window, as is sometimes necessary or desired for observation purposes.

The rim layer 30 is a compressible layer and can be made from an elastomeric material and/or a foamed material. For example, the rim layer 30 can be foamed or non-foamed rubber material. If a foamed material is employed, a closed cell foam may be used to trap air and/or moisture, or an open cell foam may be used to release air and/or moisture. Alternatively, rim layer 30 can be silicone or other elastomer ridge which conforms to the user's face.

The uniform thickness of the rim layer 30 in the pre-use relaxed state can be between about ⅛ inch and ⅜ inch. The span between the outside perimeter 36 and the inside perimeter 38 can be about one to two times the uniform thickness of the rim layer 30 in the relaxed state and/or about ⅛ inch.

As was indicated above, the inner surface 22 of the film layer 20 is bonded to the outer surface 34 of the rim layer 30. To this end, the eye-covering assembly 10 can include an adhesive 40 (e.g., a pressure sensitive adhesive) adhesively attaching the film surface 22 to the rim surface 34. The adhesive 40 can be coextensive with the outer surface 34 of the rim layer 30 to form a layer-like level between the surfaces 22/34. If rim layer comprises a silicone or other elastomer ridge, the film layer 20 can be bonded to the rim layer 30 by overmolding the rim around the film layer 20.

As was also indicated above, the inner surface 32 of the rim layer 30 adheres to facial skin during use of the covering 10, and an adhesive 50 can be provided on the rim's inner surface 34 for this purpose. This rim-to-skin adhesive 50 can be a transparent pressure sensitive water soluble gelatinous or hydrocolloidal adhesive which has sufficient stickiness to adhere appropriately during use of the eye-covering assembly 10 and which can be comfortably removed at the desired time. A rim-to-skin adhesive may or may not be necessary if the rim layer 30 comprises an elastomeric ridge.

If the eye-covering assembly 10 includes a rim-to-skin adhesive 50, it can further comprise a releasable liner 60 which covers the rim-to-skin adhesive 50. The liner 60 would prevent the adhesive 60 from accidentally sticking to another object, from accumulating dirt or debris, and/or from losing tackiness. During the application process, the liner 60 would be removed just prior to adhering the covering 10 to the user's face and discarded.

The adhesive 60 may be coextensive with the inner surface 32 of the rim layer 30. In some situations, adhesive 60 may not be necessary around the entire inner surface 32 and can be confined to selective regions, such as nose-adjacent and temple-adjacent regions. Moreover, it may be possible in some cases for the rim layer 30 to provide sufficient adherence (e.g., suction) without the use of a rim-to-skin adhesive 60. In any event, the eye-covering assembly 10 can be characterized by the absence of ties, straps, or bands which are often used to secure eye coverings to a user's face. That being said, a flexible strap may be employed to connect the left-eye cover and the right-eye cover, as a means of keeping the pair together.

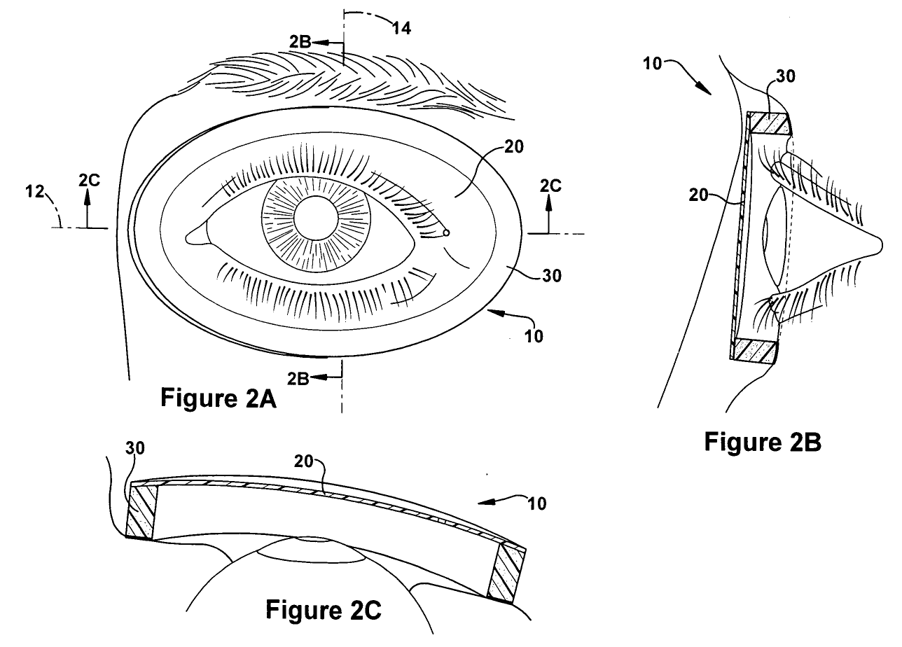

As is best seen by referring additionally to FIGS. 2A-2C, the eye-covering assembly 10 is convertible from the pre-use relaxed state to an in-use conforming state whereat it conforms to the contour of an eye-surrounding portion of a user's face. In this conforming state, the inner and outer surfaces 22/24/32/34 of the bonded film/rim layers 20/30 are curved about the nose-temple axis 12 and/or the brow-cheek axis 14. Also, the rim layer 30 can be compressed in certain regions and this compression can sometimes disrupt the uniform thickness it possesses in the relaxed state. The flexibility of the film/rim layers 20/30, and/or the compressibility of the rim layer 30, permits the eye-covering assembly 10 to accommodate a range of facial contours.

The eye-covering assembly 10 can be provided in a pre-use relaxed state whereat its flat profile facilitates stacking, rolling, dispensing, and/or storing. It may be desirable to individually package the eye-covering assembly 10 to preserve pre-use sterileness. In any event, the relaxed-to-conforming conversion can be performed by pressing the eye-covering assembly 10 against the eye-surrounding portion of the user's face. If the assembly 10 includes the adhesive 50 and the liner 60, the liner 60 can be removed prior to the converting step and/or adhering step.

After completion of the desired eye-covering period, the adherence of the inner surface 32 of the rim layer 30 can be released and the eye-covering assembly 10 removed. The film-rim layered construction of the eye-covering assembly 10 makes it suitable for single-use applications. As such, the used eye-covering assembly 10 can be disposed after removal, and a new assembly 10 used for the next eye-covering period.

If both eyes are to be covered, a pair of the eye-covering assemblies 10 can be provided, with one being applied to the right eye and the other being applied to the left eye. The pair of eye-covering assemblies 10 can be “bridgeless” in that they can be unconnected to each other. As was indicated above, if the film/rim shape is symmetrical about the brow-cheek axis 14, the same shape can be used for both the right eye and the left eye, thereby streamlining production, reducing inventory, and/or simplifying application.

The film/rim layered construction of the eye-covering assembly 10 facilitates an economical method of mass production. In this method, a sheet of film material and sheet of rim material can be used as the starting materials. The inner surface of the film-material sheet can be bonded to the outer surface of the rim-material sheet (e.g., with the film-to-rim adhesive 40). The film-material sheet and the rim-material sheet can be cut (e.g., die-cut) to form the outside perimeters 26 of the film layer 20 and the outside perimeters 36 of the rim layer 30. The rim-material sheet can be cut (e.g., die-cut) to form the inside perimeters 38 of the rim layers 30. The rim-to-skin adhesive 50 can positioned on the inner surface of the rim-material sheet and a sheet of liner-material positioned thereover.

One may now appreciate that the eye-covering assembly 10 can be economically mass produced, can be compiled in a compact manner, and/or can be applied in an uncomplicated procedure. Although the assembly 10 has been shown and described with respect to a certain embodiment or embodiments, it is obvious that equivalent alterations and modifications will occur to others skilled in the art upon the reading and understanding of this specification and the annexed drawings. In regard to the various functions performed by the above described elements (e.g., components, assemblies, systems, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function. In addition, while a particular feature may have been described above with respect to only one or more of several illustrated embodiments, such feature may be combined with one or more other features of the other embodiments, as may be desired and advantageous for any given or particular application.

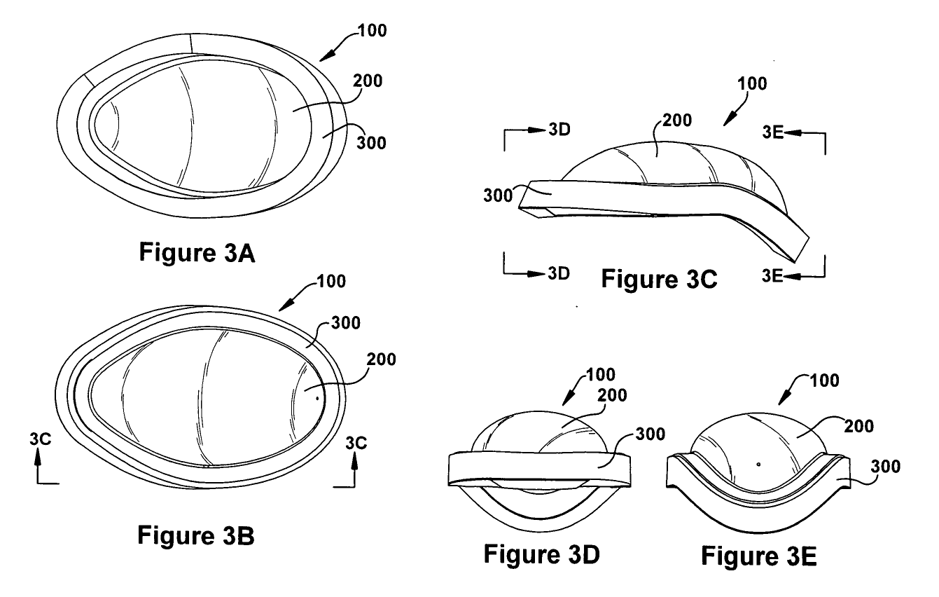

FIGS. 3A-3E show another eye-covering assembly 100 wherein a three-dimensional lens 200 is used instead of the film layer 20 in conjunction with a rim layer 300. The lens can be vacuum formed or injection molded and can be shaped to accommodate the adhering area within the eye socket. With the lens 200, a continuous arcuate geometry can be used to conform to the eye socket and it need not be flexible. The rim layer 300 can be the same or similar to the rim layer 30, and the assembly 100 can also employ a rim-to-lens adhesive, a rim-to-skin adhesive, and/or a release liner in the same manner as the assembly 10.

1. An eye-covering assembly convertible from a pre-use relaxed state to an in-use conforming state whereat it conforms to the contour of an eye-surrounding portion of a user's face; said eye-covering assembly comprising:
a uniform thickness film layer having an inner surface, an outer surface forming the assembly's exterior surface, and an outside perimeter, and
a rim layer having an inner surface for adherence to the skin of the user's face, an outer surface bonded to the inner surface of the film layer, an outside perimeter and an inside perimeter surrounding an opening;
wherein the inner and outer surfaces of the bonded film/rim layers are planar and parallel, and the rim layer has a non-compressed uniform thickness, when the eye-covering assembly is in the relaxed state; and
wherein the inner and outer surface of the bonded film/rim layers are curved about a nose-temple axis and/or a brow-cheek axis and/or the rim layer is compressed in certain regions, when the eye-covering assembly is in the conforming state.

2. An eye-covering assembly as set forth in claim 1, wherein the film layer is a one-piece layer.

3. An eye-covering assembly as set forth in claim 2, wherein the rim layer is a one-piece layer.

4. An eye-covering assembly as set forth in claim 3, wherein the outside perimeter of the film layer and the outside perimeter of the rim layer have the same shape.

5. An eye-covering assembly as set forth in claim 4, wherein the outside perimeter of the film layer and the outside perimeter of the rim layer have the same size in plan.

6. An eye-covering assembly as set forth in claim 5, wherein the film/rim layers are sized and shaped for positioning within a user's eye socket.

7. An eye-covering assembly as set forth in claim 6, wherein the film/rim layers have a dimension in the nose-temple direction of about 1¼ inches to about 2¾ inches and have a dimension in the nose-temple direction of about 1½ inches to about 2½ inches.

8. An eye-covering assembly as set forth in claim 1, characterized by the absence of straps, ties and/or bands to secure the assembly to the user's face.

9. An eye-covering assembly as set forth in claim 1, further comprising a rim-to-skin adhesive for adhering the inner surface of the rim layer to the user's face.

10. An eye-covering assembly as set forth in claim 1, wherein the uniform thickness of the film layer is between about 0.0001 inch and 0.010 inch.

11. An eye-covering assembly as set forth in claim 1, wherein the rim layer is an elastomeric layer.

12. An eye-covering assembly as set forth in claim 11, wherein the rim layer is a foam layer.

13. An eye-covering assembly as set forth in claim 11, wherein the rim layer is a molded layer.

14. An eye-covering assembly as set forth in claim 11, wherein the uniform thickness of the rim layer in the pre-use relaxed state is between about ⅛ inch and ⅜ inch.

15. A pair of the eye-covering assemblies set forth in claim 1, characterized by the absence of a strip or band connecting the pair together.

16. A pair of the eye-covering assemblies set forth in claim 1, and a flexible strip connecting the pair together.

17. A method of using the eye-covering assembly set forth in claim 1 to cover a user's eye for a desired eye-covering period, said method comprising the steps of:
providing the eye-covering assembly in its pre-use relaxed state;
positioning the eye-covering assembly so that the inner surface of the rim layer is adjacent the eye-surrounding portion of the user's face;
converting the eye-covering assembly to its in-use conforming state; and
adhering the inner surface of the rim layer to the user's skin.

18. A method as set forth in claim 17, further comprising the steps of:
releasing the adherence of the inner surface of the rim layer from the user's skin upon completion of the desired eye-covering period;
removing the eye-covering assembly from the eye-surrounding area; and
disposing of the removed eye-covering assembly after a single use.

19. A method of making a plurality of the eye-covering assemblies set forth in claim 1, said method comprising the steps of:
providing a sheet of film material having an inner surface, an outer surface, and a uniform thickness therebetween;
providing a sheet of rim material having an inner surface, an outer surface, and a uniform thickness therebetween;
bonding the inner surface of the film-material sheet to the outer surface of the rim-material sheet;
cutting the film-material sheet to form the outside perimeters of the film layers;
cutting the rim-material sheet to form the outside perimeters of the rim layers; and
cutting the rim-material sheet to form the inside perimeters of the rim layers.

20. An eye-covering assembly comprising a three-dimensional lens and a rim layer bonded to the three-dimensional len;
wherein the lens is vacuum molded and the lens is injection molded;
wherein the lens are formed in one-piece and the rim layer is a one-piece layer;
wherein the outside perimeter of the lens and the outside perimeter of the rim layer have the same shape; and
wherein the lens/rim are sized and shaped for positioning within a user's eye socket.
